(19) United States
(12) Patent Application Publication
(10) Pub. No.: US 2013/0123683 A1
(43) Pub. Date: May 16, 2013

(54) DEVICE FOR SAMPLING AND REINJECTING A FLUID, AND RELATED INJECTOR AND SAMPLING KIT

(71) Applicant: SYMATESE, Chanopost (FR)

(72) Inventor: Eric Perouse, Paris (FR)

(73) Assignee: SYMATESE, Chanopost (FR)

(21) Appl. No.: 13/664,697

(22) Filed: Oct. 31, 2012

(30) Foreign Application Priority Data
Oct. 31, 2011 (FR) 11 59876

(51) Int. Cl. A61M 5/31; A61M 5/178

(52) U.S. Cl. 604/28; 604/38

(57) ABSTRACT

A device includes a hollow body delimiting a cavity for receiving the fluid, which cavity opens in a proximal opening and at least one distal opening for sampling and/or reinjection and a piston movably mounted in the receiving cavity to obturate the proximal opening. The device includes an assembly for selective distribution of a proximal part of the fluid located in the receiving cavity, including a fluid circulation channel that passes through the piston, which channel opens into the receiving cavity. The circulation channel and/or a distribution channel is able to cooperate or to communicate with an additional flow regulation device which can be manually actionable.

### CROSS-REFERENCE TO RELATED APPLICATIONS

This application claims the benefit of French Application No. 11 59876 filed Oct. 31, 2011, which is hereby expressly incorporated by reference in its entirety.

### BACKGROUND OF THE INVENTION

1. Field of the Invention

This invention concerns a device for sampling and reinjecting a fluid, comprising:
a hollow body delimiting a cavity for receiving the fluid, which cavity opens in a proximal opening and at least one distal opening for sampling and/or reinjection;
a piston, movably mounted in the receiving cavity to obturate the proximal opening.

The fluid is advantageously a bodily fluid, such as adipose tissue of the dermis, and/or hypodermis of a human or animal. The tissue is, in particular, intended to serve as a food reserve or a protective layer against cold.

In one variant, the fluid is a biological fluid such as a blood product, a culture medium, or an element of blood.

The aforementioned device is intended, in particular, to be used to inject a fluid into a living being, e.g., to carry out a cosmetic treatment on a human. Such cosmetic treatment is advantageously an injection of bodily fluid under the skin so that it is retained and reduces or eliminates wrinkles. In one variant, the cosmetic treatment is a mammary gland reconstruction or augmentation operation, or for a fleshy mass such as a buttock.

2. Description of the Related Art

The known-art method for stretching the skin or reaugmenting its volume is to inject, under the skin, a filler product, in particular in the face or mammary glands of a human.

This type of cosmetic treatment is generally carried out using an artificial or natural product such as collagen or galuronic acid.

The use of a fluid external to the body presents certain disadvantages. In particular, the fluids injected must be perfectly sterile, and must be compatible with the human body in order to avoid causing infections, allergic reactions, or rejection by the body.

In order to remedy this issue, the known art method is to remove adipose tissue from regions of the body that have such tissue in excess, and to use the bodily fluid sampled in order to reinject it into another part of the body.

The drawing of fluid from the body is carried out, e.g., using a liposuction device comprising a needle, tubing, and a reservoir that receives the adipose tissue.

However, before reinjecting the bodily fluid into the patient, the surgeon must separate the adipose fluid into various phases in order to eliminate at least part of the fluid. To this end, surgeons remove adipose fluid from the reservoir, and then transfer the fluid to a receptacle suited for centrifuging.

They then separate the adipose fluid into several phase in order to recover a phase suitable for reinjection into the body. Surgeons must thus remove the supernatant present above the phase to be reinjected and then remove the phase to be reinjected using a syringe intended to penetrate the body of the patent.

Taking into account the injection force necessary for reinjection, the use of a mechanical injector capable of pushing the piston of the syringe with sufficient force to cause the injection of the adipose tissue is known.

Such a method of cosmetic treatment is not satisfactory. In fact, a large number of manipulations are necessary in order to remove, centrifuge, and reinject the bodily fluid, making implementation complex, in particular in the context of an operating theatre.

Additionally, taking into account the successive fluid transfers between the sampling device, the receptacle for centrifuging, and the reinjection syringe, the aseptic technique of the operation is quite debatable, and may cause infection risks.

### SUMMARY OF THE INVENTION

One objective of the invention is thus to provide a sampling and reinjection device for bodily fluid, in particular adipose tissue, which device is particularly simple and safe to operate.

To this end, the invention provides a device of the aforementioned type, characterised in that the device includes a selective distribution assembly for a proximal part of the fluid located in the receiving cavity, which distribution assembly includes a fluid circulation channel running through the piston, opening into the receiving cavity.

The device according to the invention may comprise one or more of the following characteristics, taken alone or in all combinations technically possible:
the piston comprises a distal body to obturate the cavity and a drive rod to actuate the body, whereby the circulation channel passes through the distal body and through the rod;
the distribution assembly comprises a proximal distribution tap connected to the circulation channel opposite the receiving cavity, which proximal tap can be moved together with the piston;
it comprises an auxiliary connector to evacuate the receiving cavity, which connection opens into the receiving cavity apart from the distal opening;
it comprises an evacuation line intended to be connected on the one hand to the receiving cavity, and, on the other, to a source of negative pressure.
it comprises at least one detachable sampling and/or reinjection line intended to be connected to the receiving cavity via the distribution assembly;
the proximal end of the hollow body is equipped with locking means suited to be received by the supplementary blocking means of a centrifuge;
it comprises at least one bladder receiving in its interior volume at least one first nozzle connected to the receiving cavity and at least one second nozzle of a line, whereby the first nozzle can be connected reversibly to the second nozzle.

The invention further concerns kit for sampling and reinjecting a fluid, characterised in that it comprises
a device as described above;
an injector comprising at least one actuator of the piston intended to move the piston within the receiving cavity.

The kit according to the invention may comprise one or more of the following characteristics, taken alone or in all combinations technically possible:
it comprises a vacuum pump intended to be connected hydraulically to the filling cavity;
it comprises a centrifuge, which centrifuge comprises a housing for receiving the sampling device;
the injector comprises:
a retainer bracket for the hollow body of the device;
an actuator mounted movably with respect to the bracket, which actuator is intended to cooperate with the piston to move the piston in the receiving cavity;
the actuator comprises a housing to receive the distribution assembly and/or a detachable line intended to be connected to the fluid circulation channel.
the actuator comprises a rod that can be moved axially through the bracket, whereby the housing opens axially into a support surface on the piston and transversally through a lateral surface of the rod.

The invention also concerns an injector intended to receive a device as described above, which injector comprises:
a retainer bracket for the hollow body of the device;
an actuator mounted movably with respect to the bracket, which actuator is intended to cooperate with the piston to move the piston in the receiving cavity;

characterised in that the actuator comprises a housing to receive the distribution assembly and/or a detachable line intended to be connected to the fluid circulation channel.

The injection according to the invention may comprise one or more of the following characteristics, taken alone or in all combinations technically possible:
the actuator comprises a rod that can be moved axially through the bracket, whereby the housing opens axially into a support surface on the piston and transversally through a lateral surface of the rod.

The invention also concerns a non-therapeutic cosmetic method for treating a living being, comprising the following steps:
provision of a device as described above, whereby the receiving cavity of its body contains a fluid, advantageously a non-therapeutic fluid;
selective evacuation of a proximal part of the fluid present in the receiving cavity, whereby the piston obturates the proximal opening, independently of a distal part of the fluid located between the proximal part and the distal opening via the distribution assembly, the proximal part being evacuated through the evacuation channel arranged in the piston.

The method according to the invention may comprise the following characteristic:
it includes a step of separating the fluid contained in the receiving cavity into a first proximal phase and at least one second distal phase, which method comprises the following steps:
evacuation of the first proximal phase from the receiving cavity through the distribution assembly without evacuating the second distal phase;
it includes a step of filling the cavity with a bodily fluid.

### BRIEF DESCRIPTION OF THE DRAWINGS

The invention will be better understood based on the following description, provided by way of example only, referring to the attached drawings, in which :

FIG. 1 is a schematic side view of a first sampling and reinjection kit for bodily fluid according to the invention;

FIG. 2 is a schematic view of a partial cross section of a sampling and reinjection device before use;

FIG. 3 is a top view of detail marked III on FIG. 1;

FIG. 4 is a view analogous to FIG. 3 in a three-quarter face perspective;

FIG. 5 is a view analogous to FIG. 2 during a step of sampling bodily fluid from a patient;

FIG. 6 is a view analogous to FIG. 5, during a step of eliminating at least part of the bodily fluid; and

FIG. 7 is a view analogous to FIG. 5 during a step of reinjecting the bodily fluid into a human;

FIG. 8 shows a second kit according to the invention;

FIG. 9 shows a third kit according to the invention; and

FIG. 10 shows a fourth kit according to the invention;

FIG. 11 is a view analogous to FIG. 7 for a fifth kit according to the invention;

FIG. 12 is a detail view of the kit of FIG. 11, illustrating an additional flow regulator device.

### DETAILED DESCRIPTION OF THE PREFERRED EMBODIMENT

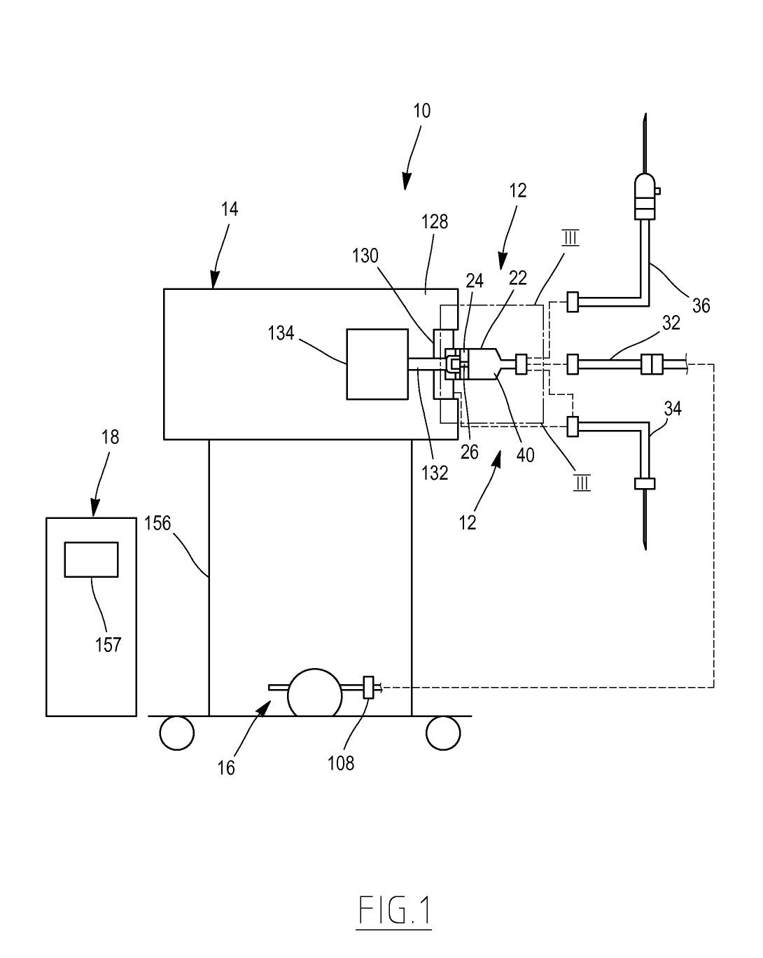

A first kit 10 for sampling and reinjecting fluid according to the invention is schematically illustrated in FIG. 1.

This kit 10 is advantageously used to implement a cosmetic, non-therapeutic treatment method on a human or animal subject.

The kit 10 comprises a first sampling and reinjection device 12, a mechanical injector 14, a vacuum pump 16, and a centrifuge 18.

The bodily fluid to be sampled and reinjected by the device 12 is, e.g., adipose tissue from the dermis and/or hypodermis of a human or animal, in particular that having the double role of food reserve and protective layer against cold. Such adipose tissue is generally known as “subcutaneous fat”. This fluid, once sampled, is suited to be separated into various phases, of which at least one phase is suitable for reinjection by the device 12 according to the invention.

In one variant, the fluid is a biological fluid such as a blood product, a culture medium, or an element of blood.

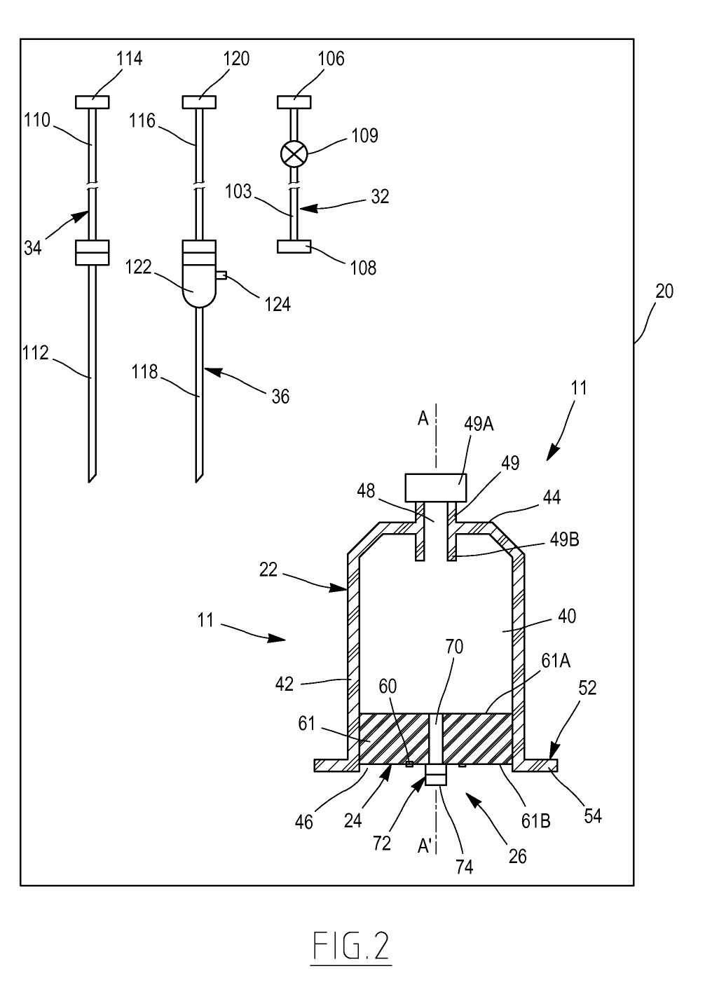

As shown by FIG. 2, the device 12 is preferably initially contained in sterile packaging 20.

It includes an assembly 11 of a single piece including a hollow syringe body 22, a piston 24 mounted movably in the body of the syringe 22, and, according to the invention, and a distribution assembly 26 borne by the piston 24.

In the example shown in FIG. 2, the device 12 additionally includes a detachable evacuation line 32, a first detachable bodily fluid sampling line 34, and a second detachable bodily fluid reinjection line 36.

The syringe body 22 is made, e.g., in a single part and of a transparent material, such as glass or plastic. It is intended to be connected, in a first sampling step, to the vacuum pump 16 and the sampling line 34 to sample the bodily fluid from the subject.

The body 22 is then mounted in a housing of the centrifuge 18 in order to separate the fluid sampled in several phases. The body 22 is additionally connected to the injector 14 and the reinjection line 36 in order to allow the reinjection of at least one phase of the bodily fluid into the subject.

The body 22 is tubular in shape, and extends along an axis A-A′ shown vertically in FIG. 2. Internally, it forms a cavity 40 for receiving the bodily fluid. It includes a peripheral wall 42 and a distal wall 44, distally obturating the peripheral wall 42.

The cavity 40 is delimited externally by the peripheral wall 42 and distally by the distal wall 44. It opens upward by means of a main proximal opening 46 located in the proximal end of the wall 42.

It opens downward by means of a distal sampling and reinjection opening 48.

The distal opening 48 is located in the distal wall 44. It is axially opposite the proximal opening 46. The distal opening 48 has a cross section smaller than the cross section of the main opening 46.

The body 22 has a distal connection 49, which projects from the distal wall 44 around the distal opening 48. The distal connection 49 has a connection nozzle 49A to be connected movably to a line 32, 34, 36. The nozzle 49A is also suited to receive a selective obturator of the opening 48, such as a plug or a butterfly valve.

Advantageously, the distal connection 49 includes a proximal section 49B projecting into the cavity 40 to guide the evacuation of fluid through the opening 48.

The volume of the receiving cavity 40 may range, for example, between 5 cm3 and 300 cm3.

In the vicinity of the proximal opening 46, the lateral wall 42 is equipped on the outside with means 52 for locking the body 22 on the injector 14 and on a housing of the centrifuge 18. These means 52 are formed e.g., by threading or by a securing flange 54 schematically represented in FIG. 2.

The piston 24 has an external cross section complementary with the internal cross section of the cavity 40. It hermetically seals the cavity 40 in a proximal direction.

As shown in FIG. 2, the piston 24 is advantageously equipped with means of hanging 60, to be engaged with an mobile actuator present in the injector 14.

The piston 24 is mounted movably in the cavity 40 along the axis A-A′, between a proximal position, located in the vicinity of the proximal opening 46, and a distal position, located in the vicinity of the distal wall 44.

When moved along the axis A-A′, the piston 24 hermetically seals the cavity 40 with the exception of the distribution assembly 26. This prevents the fluid present in the cavity 40 escaping through the proximal opening 46, save to pass through the distribution assembly 26.

As shown in FIG. 2, the piston 24 includes an obturator 61, having an external cross section that is complementary with the internal cross section of the cavity 40.

The piston 24 has an internal surface 61A arranged in the cavity 40 and an opposite external surface 61B, in which the means of hanging 60 are located, if present.

The external surface 61B is intended to come into contact with the actuator present on the injector 14.

According to the invention, the distribution assembly 26 includes a fluid circulation channel 70, passing through the piston 24, and, advantageously, a proximal connecting tap 72.

In the example shown in FIG. 2, the circulation channel 70 is positioned axially through the body 61 of the piston 24. It opens into the internal surface of 61A, and the external surface 61B. It fluidly connects the cavity 40 to the proximal tap 72.

The proximal tap 72 projects externally from the external surface 61B. It is connected to the circulation channel 70. It is movably mounted jointly with the piston 24 when the piston 24 is moved.

The proximal tap 72 is equipped with a proximal nozzle 74 to be connected selectively to a line 34, 36, or to an obturator formed, e.g., by a plug or butterfly valve.

In this example, the channel 70 is essentially parallel to the axis A-A′, coaxially with the axis A-A′. In one variant, the channel 70 is staggered transversely with the axis A-A′ and/or extends on an incline with respect to the axis A-A′.

The evacuation line 32 is formed, e.g., based on rigid tubing 103. It includes an upward opening 106 to be mounted on the distal nozzle 49A of the distal connection 49, and a downward opening 108 to be connected to the vacuum pump 16. The evacuation line 32 is advantageously equipped with a permeate valve 109.

As shown in FIG. 2, the sampling line 34 includes connecting tubing 110, which is advantageously flexible, and a sampling needle 112 more rigid than the tubing 110. The needle 112 is formed, e.g., by a liposuction needle. The tubing 110 has an upward nozzle 114 to be connected to the nozzle 74 of the proximal tap 72 and/or the nozzle 49A of the distal connection 49.

The reinjection line 36 comprises flexible reinjection tubing 116 equipped on the downward end with a reinjection needle 118 more rigid than the tubing 116. In FIG. 2, the reinjection line 36 is advantageously distinct from the sampling line 34. In one variant, the line 34 simultaneously forms the sampling line and the reinjection line.

The reinjection line 116 has a nozzle 120 to be connected to the nozzle 49 A of the distal connection 49 and/or the nozzle 74 of the proximal tap 72.

In an advantageous variant, a module 122 to control the rate of flow of the reinjected bodily fluid is arranged on the reinjection line 36. This module 122 includes a controller 124 that can be controlled by an operator to control the flow circulating through the tubing 116 and through the reinjection needle 118.

In one variant, the control module 112 is arranged upstream of the reinjection line 36, between the distal connection 49 and the nozzle 114, whereby the controller 124 can be displaced with respect to the module 122.

The controller 124 is, e.g., a button, as shown in FIG. 2. In one variant, the controller 124 is a pedal, in case the operator must use both hands.

Each of the lines 32, 34, 36 is intended to be mounted removably and reversibly respectively on one and/or the other of the distal connection 49 and the proximal tap 72.

With reference to FIGS. 1, 2, and 3, the injector 14 according to the invention includes a frame 128, a bracket 130 for receiving the syringe body 22, an actuator 132 for the piston, and a housing 133 to receive the actuator 132.

The injector 14 additionally comprises means of control 134 of the movement of the actuator 132.

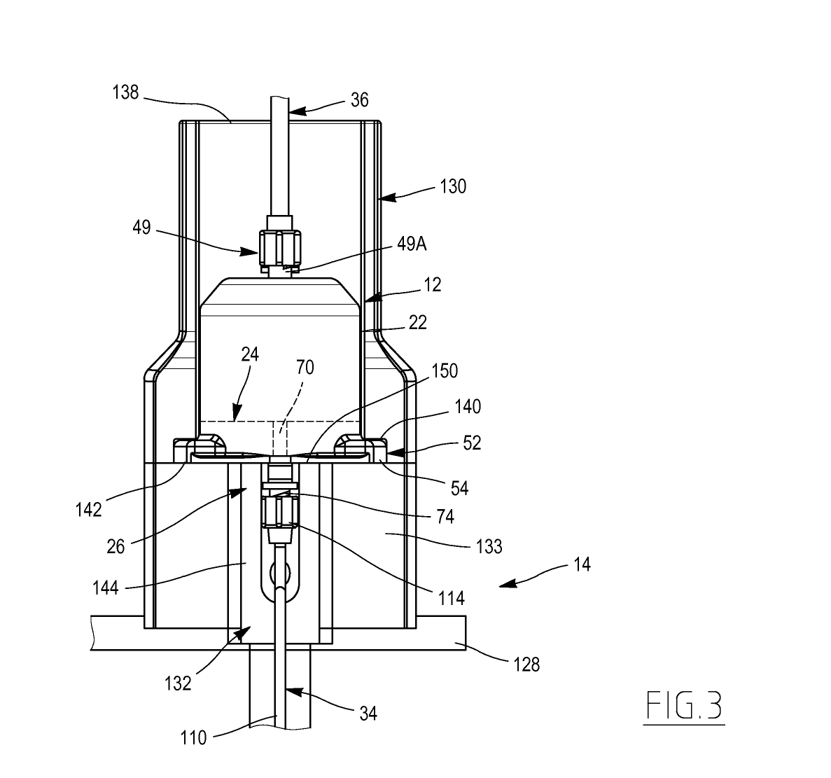

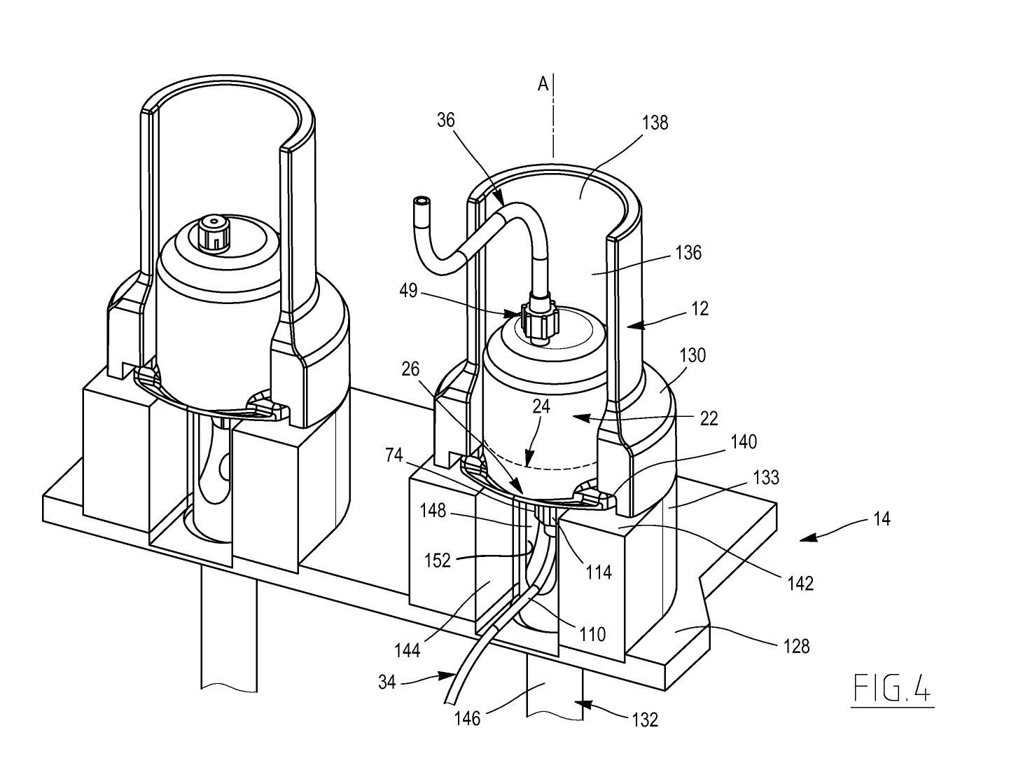

As shown in FIGS. 3 and 4, the bracket 134 is formed, e.g., by a half-shaft mounted on the housing 133. The bracket 130 thus advantageously has a lateral opening 136 into which to insert the body 22 and a superior opening 138 through which a line 34, 36 passes.

The bracket 130 has means of blocking in addition to the locking means 52 of the body 22 arranged in the vicinity of the proximal opening 46.

The additional means of blocking are located in the bracket 130. They are formed, e.g., by grooves 140, forming a retaining flange for the locking means 52.

The body 40 may thus be inserted into the bracket 130 via the lateral opening 136 by removably engaging the locking means 52 with the grooves 140.

The housing 133 receiving the actuator is arranged between the frame 128 and the bracket 130. It delimits a superior surface 142 supporting the body 40 and a central lumen 144 through which the actuator 130 passes.

The lumen 144 opens opposite the bracket 130. It opens in a lateral passage located under the lateral opening 136 to allow the passage of the proximal tap 72, and, if applicable, of a line 32, 34, 36 during the insertion of the body 22 into the bracket 130.

The actuator 132 includes a rod 146 delimiting a housing 148. The housing 148 is intended to receive the proximal tap 72 and, if applicable, a line 32, 34, 36.

The rod 146 extends through the frame 128, through the housing 133, and through the bracket 130 when deployed with respect to the frame 128. It has a superior support surface 150 on the piston 24. The superior surface 150 is located on its free end.

The superior surface 150 is intended to cooperate with the means of hanging 60 to move the piston 24 in the cavity 40.

In this example, the housing 148 is formed by an axial notch.

More generally, the housing 148 opens axially following the axis of the rod 146 in the superior surface 150 through an axial opening intended to extend facing the external surface 61B of the piston 24.

The housing 148 opens transversely through a lateral opening 152, allowing the passage of a line 34, 36 and the insertion of the proximal tap 72. The lateral opening 152 extends facing the lateral passage through which the lumen 144 opens.

The actuator 132 is thus movable along an axis A-A′ in the housing 133 and in the bracket 130 between a retracted position in the housing 133 and a deployed position in the bracket 130, via the control 134.

When moved, the actuator 132 moves the piston 24 from its proximal position to its distal position.

The actuator 132 jointly moves the proximal tap 72 and, if applicable, the line 32, 34, 36 mounted on the opening 74 of the tap 72.

Thus, the syringe body 22 may be mounted reversibly on the bracket 130 on the injector 14. Once mounted on the injector 14, the actuator 132 is suited to cooperate with the means of engagement 60 on the piston 24 to move the piston 24 from its proximal position to its distal position at a controlled speed.

The vacuum pump 16 is suited to create a vacuum, e.g., on the order of −950 mbar relatively in the receiving cavity 40 when the evacuation line 32 is connected simultaneously to the vacuum pump 16 and the distal connection 49, and when the butterfly valve 109 is open.

In an advantageous variant, shown in FIG. 1, the injector 14 and the vacuum pump 16 are received on the same chassis 156 so that they can be moved jointly.

The centrifuge 18 includes at least one housing 157 (shown in FIG. 1) suited to receive in a fixed manner the syringe body 22, the sampling assembly 26, and the distal connection 49.

The housing 157 has a receiving housing (not shown) to receive the proximal end of the syringe body 22 and the locking means 52.

The housing of the housing 157 is equipped with means of blocking in addition to the locking means 52. It additionally includes a cap to hold the piston 24 in position in order to prevent it moving through the proximal opening 46.

Once received by the housing 157, the syringe body 22 is locked in place reversibly, and is suited to be rotated in order to cause a phase separation within the fluid contained in the receiving cavity 40.

The operation of the sampling kit 10 according to the invention in the case of a non-therapeutic cosmetic treatment operation on a living being will be described below.

Initially, referring to FIG. 2, a reinjection and sampling device 12 is provided, advantageously contained in sterile packaging 20.

The operator then opens the packaging 20 and extracts the syringe body 22, the piston 24, and the sampling assembly 26.

The operator then connects the evacuation line 32 to the distal connection 49, and opens the butterfly valve 109 to release the tubing 103. The sampling line 34 is then connected to the proximal tap 72.

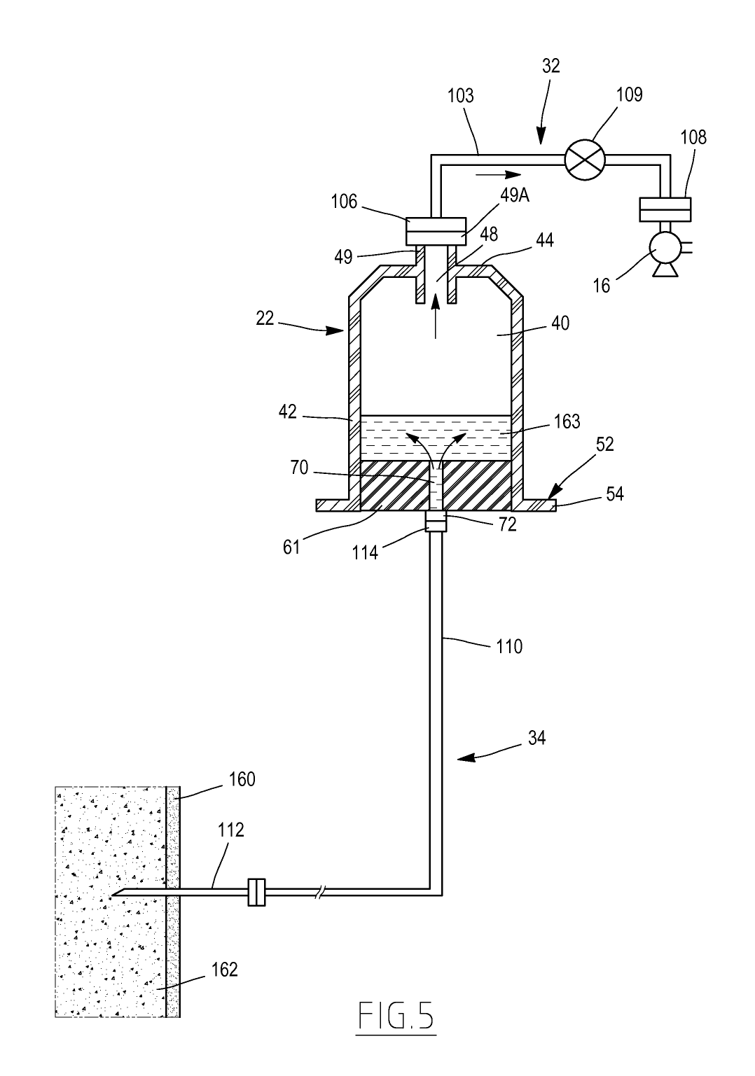

Once connected (FIG. 5), the sampling line 34 is held by the operator. The operator then penetrates the skin 160 of the subject receiving the cosmetic treatment with the needle 112 to draw adipose tissue 162 under the skin 160.

The vacuum pump 16 is then activated, causing negative pressure within the cavity 40 via the evacuation line 32 and the distal opening 48.

The piston 24 initially occupies its proximal position in the vicinity of the proximal opening 46.

Under the negative pressure, fluid 163 is aspirated successively through the sampling needle 112, the sampling tubing 110, the proximal tap 72, and the circulation channel 70 in order to progressively fill the cavity 40 from bottom to top, as shown in FIG. 5.

Once the cavity 40 is at least partially filled, the operator disassembles the sampling line 34 and the evacuation line 32. The distal connection 49 and the proximal tap 72 are then plugged, e.g., using a plug or butterfly valve (not shown).

The operator then takes the syringe body 22 obturated in the upward direction by the piston 24, and inserts the proximal end of the body 22 into a housing 157 of the centrifuge 18.

The operator then engages the locking means 52 in the housing to hold the body 22 in position, and to lock the piston 24 in the housing 157. The piston 24 is then axially immobilised in the housing 157.

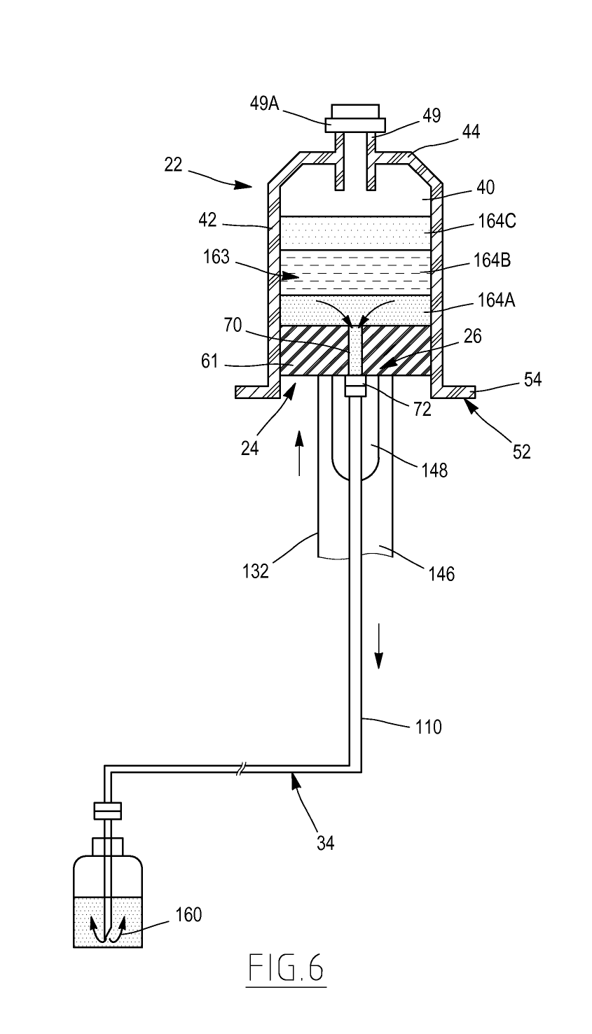

The operator then activates the centrifuge 18 to separate the fluid 163 contained in the receiving cavity 40 into various phases 164A, 164B, 164C shown in FIG. 6. This separation is obtained by rotating the housing 157 containing the body 22.

Once the separation operation has been carried out, the operator disassembles the syringe body 22 to extract it from the centrifuge 18, releasing the locking means. Then, as shown in FIG. 5, the operator connects an evacuation line, e.g., formed by the sampling line 34, to the proximal tap 72.

The body 22 is then positioned vertically on the injector 14 to eliminate the lower and upper phases 164A, 164C, respectively, saving only an intermediate phase 164B for reinjection into the subject.

To this end, the body 22 is inserted into the bracket 130. Referring to FIGS. 3 and 4, the proximal tap 72 and part of the line 34 transversely penetrate the housing 148 positioned at the free end of the rod 146 via the lateral opening 152 and the lumen 144.

As shown in FIG. 4, the upward nozzle 114 of the tubing 110, the proximal tap 72, and the upstream part of the tubing 110 are then received by the housing 148. Part of the tubing 110 then projects out of the housing 148 through the lumen 144, and outside of the housing 133.

During this operation, the distal connection 49 remains blocked.

Then, as shown in FIG. 6, the operator activates the controller 134 of the injector 14 to move the piston 24 along the axis A-A′ towards the distal end of the body 22.

During this movement, the lower phase 164A is successively evacuated through the channel 70, the tap 72, and then through the evacuation line into a container 160 for disposal.

Then, once the lower phase 164A has been eliminated, the operator blocks the proximal tap 72. An evacuation line is mounted, e.g., the sampling line 34, on the distal connection 49 of the body 22.

The operator then once again activates the controller 134 to move the piston 24 towards the distal end of the body 22, thus evacuating the upper phase 164C through the distal connection 49, and then through the evacuation line 34.

Once this is done, the operator detaches the evacuation line and mounts the reinjection line 36 onto the distal connection 49. The superior surface of the intermediate phase 164B is then located at the level of the proximal section 49B of the distal connection 49.

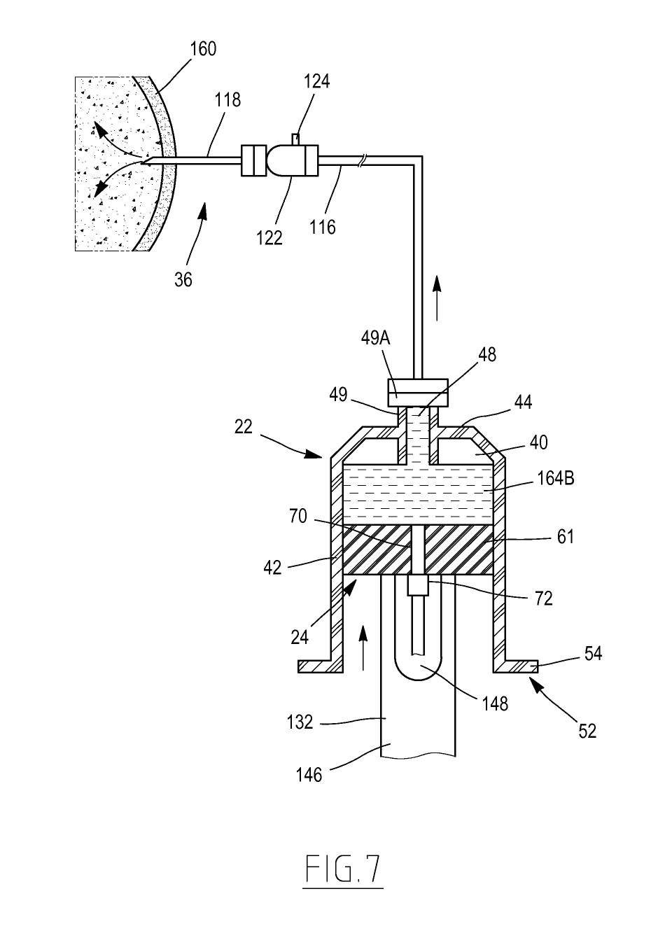

Then, referring to FIG. 7, the operator penetrates the skin 160 of the subject in a region other than that from which the adipose tissue 162 was drawn, e.g., in the face, mammary glands, or a fleshy part.

The operator then activates the controller 124, activating the means of control 134 and the movement of the actuator 132, to push the piston 24 towards the distal end of the body 22. This causes the evacuation of the selected phase 164B of the bodily fluid present in the cavity 40, successively through the distal connection 49, the reinjection tubing 116, the control connection 112, and the reinjection needle 118.

The device 12 according to the invention thus allows for all operations necessary for the sampling, centrifuging, and reinjection of the fluid to be carried out successively, without having to change the fluid of the receptacle. The aseptic technique of the operation is reinforced, and the risk of microbial contamination is very low.

Furthermore, the device 12 is extremely simple to handle, in particular in the environment of an operating room. In particular, the presence of the distribution assembly 26 joined with the piston 24 allows for the elimination of all phases of the fluid that are not desired so that only one or more selected phases are injected.

The device 12 thus allows for clean, simple handling of the bodily fluid without risk of leakage or contamination.

More generally, the bodily fluid drawn from a first living being is not necessarily reinjected into this living being, but instead reinjected into a second living being.

Thus, the foregoing shows that the distribution assembly 26 is suited for selective evacuation of an upstream part of the fluid present in the receiving cavity 40, independently of a downstream part of the fluid located between the upstream part and the or every distal opening 48.

The upstream part of the fluid is advantageously formed by the lower phase 164A, which may be evacuated through the distribution assembly 26, independently of the intermediate phase 164B, and without having to extract the intermediate phase 164B through a distal opening 48.

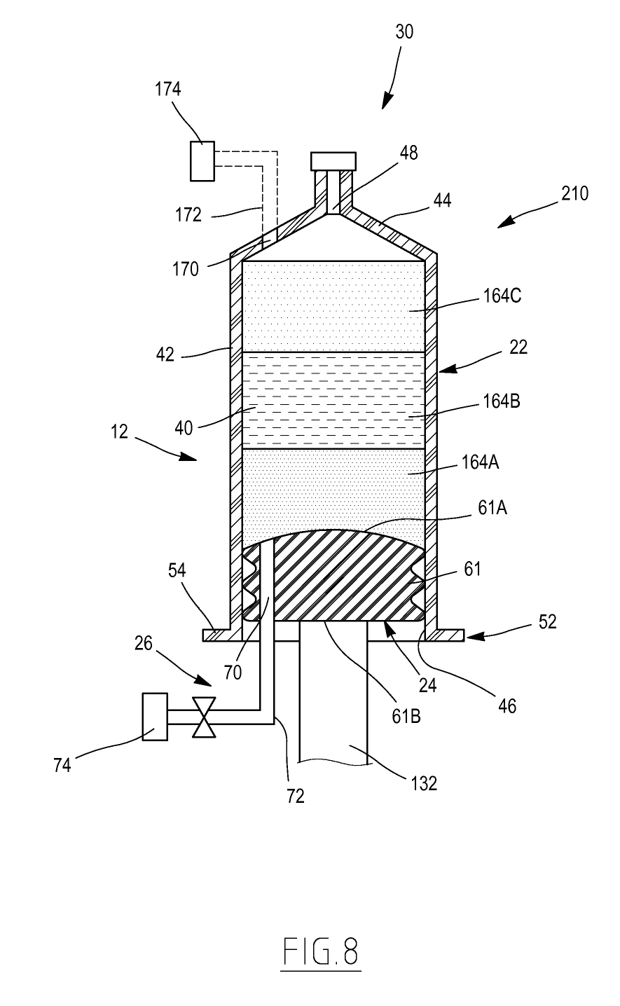

A second kit 210 according to the invention is shown in FIG. 8. This kit 210 is analogous to the first kit 10.

As shown in FIG. 8, the sampling and reinjection device 12 of the second kit 210 differs from the device 12 of the first kit 10 due to the characteristic that the distribution assembly 26 is axially staggered with the axis of the piston 24.

The operation of the second kit 210 is analogous to that of the first kit 10.

The device 12 can then be mounted on an injector 14 having an actuator 132 that is not necessarily equipped with a housing 148 to receive the proximal tap 72.

The proximal tap 72 projects away from the actuator 132 with respect to the piston 24.

In one variant of the device 12, shown in dotted lines in FIG. 8, the body 22 includes an additional distal opening 170 distinct from the distal opening 48. The additional distal opening 170 opens into an auxiliary connection 172 mounted on the body 22 and equipped with a nozzle 174 for connection to a line 32, 34, 36.

Thus, the auxiliary opening 170 can be connected to the evacuation line 32, and the main opening 48 can be connected to the sampling line 34 and/or the reinjection line 36. This variant also applies to the first kit 10.

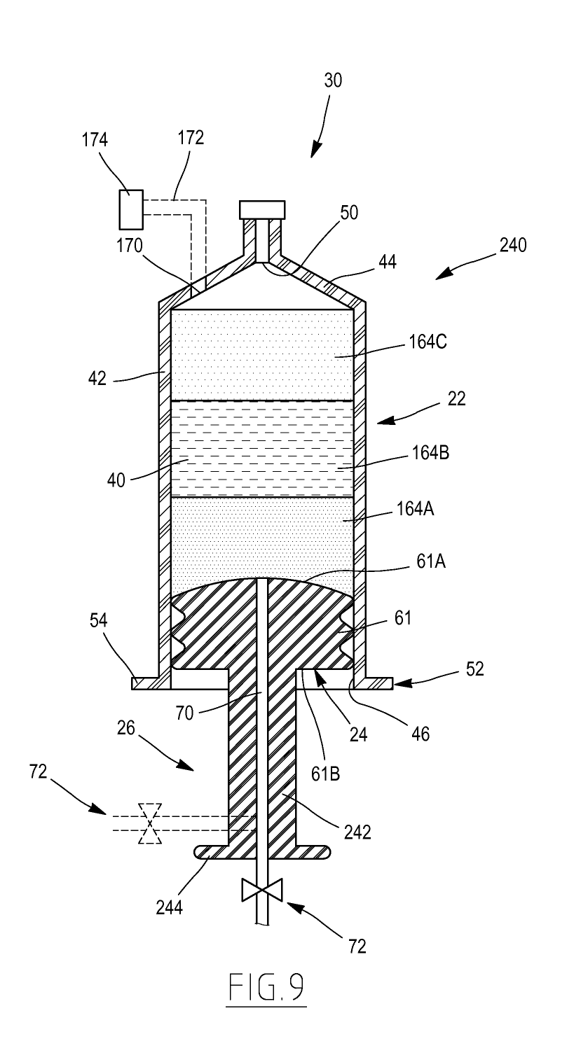

A third kit 240 according to the invention is shown in FIG. 9.

Unlike the second kit 210, the piston 24 includes, in addition to the obturation body 61, a proximal rod 242 actuating the piston 24, which projects externally from the obturation body 61. In this example, the channel 70 extends successively through the body 61, then through the rod 242, to open outside of the rod 242, advantageously at the level of the proximal end 244 of the rod 242. The proximal tap 72 is connected to the rod 242.

In the device shown in FIG. 9, the proximal connection 72 projects axially on the axis of the rod 242 of the piston.

In one variant, shown in dotted lines, the proximal connection 72 projects transversely with respect to the axis of the rod 242. The channel 70 is then bent.

The operation of the third kit 240 is otherwise analogous to that of the first kit 10.

In one variant, the piston 24 is not actuated by an injector 14. It is actuated manually by the fingers of an operator.

This applies, in particular, to small syringes having a volume less than 50 ml, in particular ranging between 5 ml and 15 ml, or sampling tubes of the same volume to evacuate a supernatant without contaminating the phase(s) that are to be saved.

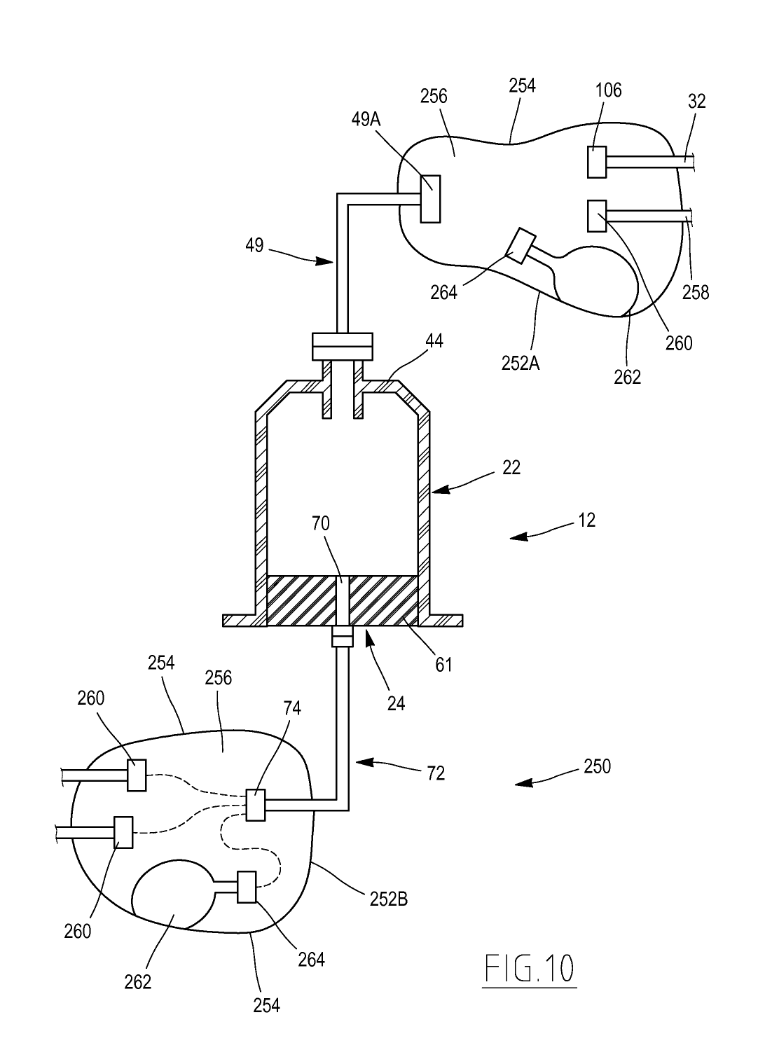

A fourth kit 250 according to the invention is shown in FIG. 10. Unlike the device 12 of the first kit 10, the device 12 of the fourth kit 250 includes at least one protection bladder 252A, 252B to ensure aseptic technique during the connection of the lines 32, 34, 36 respectively to one or the other of the distal connection 49 and the proximal tap 72.

The device 12 shown in FIG. 10 advantageously includes a first bladder 252A mounted on the distal connection 49, and a second bladder 252B mounted on the proximal tap 72.

Each bladder 252A, 252B is formed by a soft envelope 254, advantageously transparent. The soft envelope 254 delimits an internal volume 256 of gas that is substantially hermetically sealed.

The envelope 254 of the first bladder 252A is mounted hermetically around a distal part of the distal connection 49. It receives the nozzle 49A of the distal connection 49 in its internal volume 256.

The first bladder 252A is also mounted hermetically around the proximal part of the evacuation line 32. It is advantageously mounted hermetically around a proximal end of an evacuation line 258.

The volume 256 thus advantageously receives the upward nozzle 106 of the evacuation line and the upward nozzle 260 of the evacuation line 258.

In an advantageous variant, the bladder 252A additionally contains at least one pocket 262 for recovering a phase to be eliminated, equipped with a nozzle 264 at its end.

Thus, the nozzle 49A of the distal connection 49 can be connected selectively to one of the nozzles 106, 260, 264 contained in the bladder 252A during the various phases of the procedure. This operation can be carried out without the operator having to touch the nozzles 106, 260, 264, by simply twisting the envelope 254.

Likewise, the envelope 254 of the second bladder 252B contains the nozzle 74 of the proximal tap. This nozzle 74 can be connected selectively to a nozzle 260 of an evacuation line 258 or a nozzle 264 of a pocket 262, whereby the nozzles 74, 260, 264 are received in the envelope 264 of the second bladder 252B.

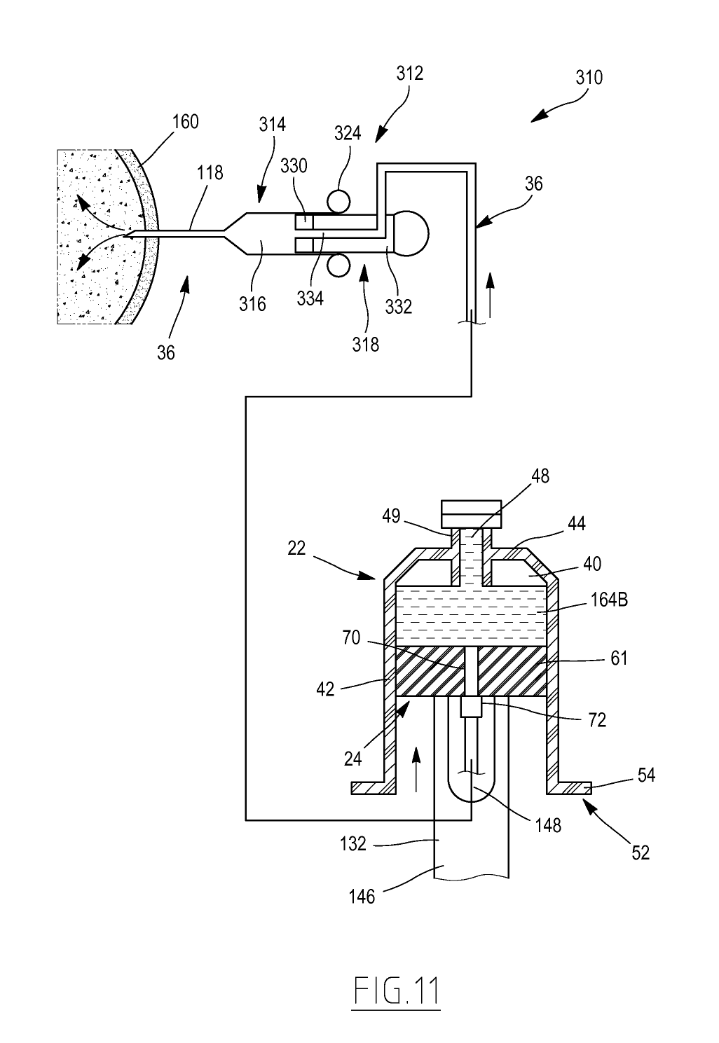

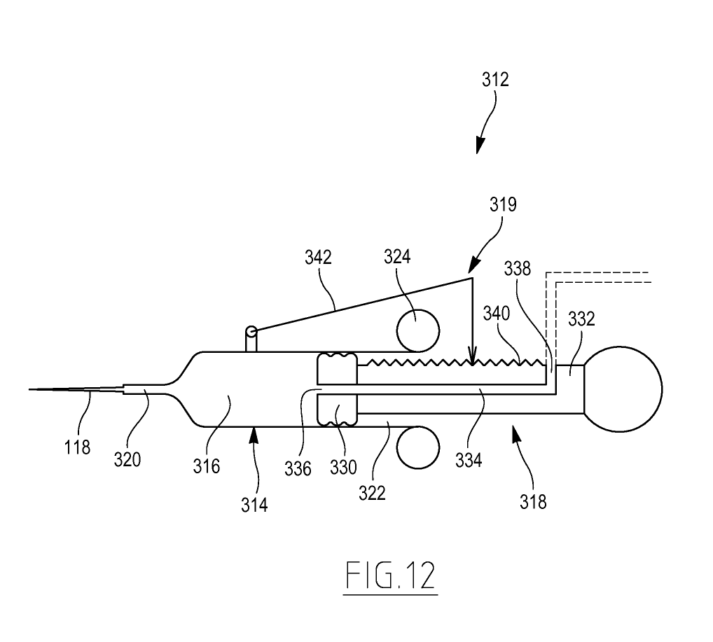

A fifth kit 310 according to the invention is shown in FIGS. 11 and 12.

Like the first kit 10, the fifth kit 310 includes a first sampling and reinjection device 12 to be mounted removably on a mechanical injector 14.

The fifth kit 310 according to the invention differs from the first kit 10 in that it includes an additional 312 for manual adjustment of the flow rate of the fluid reinjected into the subject.

The device 312 is connected to the reinjection apparatus 118, which is advantageously a needle or a fine cannula.

In this example, the additional device 312 is formed by a syringe that carries the reinjection apparatus 118.

As shown in FIG. 12, the device 312 includes an additional hollow body 314 delimiting an internal volume 316 to regulate the flow. The device 312 additionally includes a mobile piston 318 in the body 316 to block the internal volume 316 upstream.

In this example, the device 312 additionally advantageously includes a releasable mechanism 319 to lock the piston 318 in position with respect to the body 314.

The additional hollow body 314 is tubular in shape. It delimits a distal opening 320 connected to the needle 118, and a proximal opening 322 through which the piston 318 is inserted into the body 314.

The body 314, around its proximal opening 322, includes a flange 324 suited to be held by the fingers of a user to hold the hollow body in position 314.

The piston 318 includes a head 330 to obturate the volume 316, inserted in the body 114, and a drive rod 332, suited to project outside of the body 314 to be pushed by a finger of the user.

The piston 318 internally delimits a channel 334 to carry fluid into the volume 316. The channel 334 opens downward in a downstream orifice 336 located in the volume 316, and upward by an upward orifice 338 located outside of the body 314.

The upward orifice 338 is intended to be connected to the fluid reinjection line 36. In the example shown in FIG. 12, it opens transversely with respect to an axis of the piston 318.

In this example, the mechanism 319 includes a locking shaft 340 on the piston 318, and a mobile apparatus 342 for engaging the shaft 340, mounted on the body 314.

The engaging apparatus 342 can move from a position in which the piston 318 is released, in which it is located away from the shaft 340, and various positions in which the piston 318 is locked with respect to the body 314, in which it is engaged with at least one tooth of the shaft 340.

In the example shown in FIG. 11, the reinjection line 36 is connected upstream to the circulation channel 70 located in the piston 24. It is housed at least partially in the housing 148 located in the rod 146 of the actuator 132.

The operation of the fifth kit 310 according to the invention is analogous to that of the first kit 10 until the end of the step in which the phases 164A, 164C that are not wanted for reinjection into the subject are separated and eliminated.

The operation of the fifth kit 310 according to the invention differs from the operation of the first kit 10 at the reinjection step.

Before this step, the operator connects the reinjection line 36 to the body 22 of the device 12. In this example, the upstream end of the line 36 is mounted on the connection 72 to connect the channel 70 to the line 36. Additionally, the downstream end of the reinjection line 36 is affixed to the piston 38 of the additional device 312 to connect the reinjection line 36 to the channel 334. The distal connection 49 is additionally plugged.

Initially, the operator fills the internal volume 316 of the additional device 312. To this end, the operator activates the injector 14, causing the rod 146 of the actuator 132 to move towards the inside of the volume 40 to push the piston 24.

This movement of the piston 24 causes the evacuation of the fluid 164B through the line 36, and through the channel 334 until the internal volume 316.

When the internal volume 316 is filled, the operator penetrates the skin 160 of the subject.

In a first mode of operation, when an injection of fluid at a constant flow rate is required, the operator adjusts the constant speed of movement of the actuator 132, maintaining in place the piston 318 of the additional device 312 with respect to the body 314.

It is held in place manually, or using the mechanism 319. The internal volume 316 remains constant.

In this mode of operation, a constant flow of fluid is injected into the subject automatically by the injector 14.

In a second mode of operation, when the flow of the fluid injected must be slightly increased, the operator manually moves the piston 318 of the additional device 312 towards the downstream opening 320 to reduce the volume 316. This movement of the piston 318 occurs without changing the flow setting of the injector 14.

A greater flow rate of fluid is then ejected slightly outside of the additional device 312, allowing, e.g., for rapid filling of a bodily cavity or area in order to increase its volume.

In a third mode of operation, when the flow rate of the fluid injected must be slightly reduced, the operator releases the piston 318 of the additional device 312, leaving it free to move away from the downstream opening 320. This causes an increase in the intermediate volume 316 and the corresponding decrease of the rate of flow of the fluid extracted through the needle 118. In one variant, the operator manually moves the piston 318 away from the downstream opening 320.

Such a mode of operation allows for precise control of the amount of fluid extracted from the additional device 312, in particular for a precise injection of additional fluid into the subject.

The presence of an additional device 312 thus allows for precise regulation of the amount of fluid extracted from the kit 310 in order to reinject it into the subject. The adjustment is made manually by simply actuating the piston 318 using the operator's fingers without having to adjust the setting of the injector 14.

Thus, in normal operating conditions, the operator keeps the piston 318 in position. The amount of fluid injected is then controlled directly by the injector 14. This involves no effort on the part of the operator.

When additional fluid must be added more quickly and precisely, a simple manual manipulation of the piston 318 allows for precise control of the amount of fluid injected without having to modify the setting of the injector 14. This is also the case if the flow rate of the fluid ejected from the kit 310 must be slightly reduced, in particular in an area in which the injection must be very precise.

What is claimed is:

1. A kit for sampling and reinjecting a fluid comprising:
a device for sampling and reinjecting a fluid, comprising:
a hollow body delimiting a cavity for receiving the fluid, which cavity opens in a proximal opening and at least one distal opening for sampling and/or reinjection;
a piston, movably mounted in the receiving cavity to obturate the proximal opening;
wherein the device comprises an assembly for selective distribution of a proximal part of the fluid located in the receiving cavity, comprising a fluid circulation channel that passes through the piston and opens into the receiving cavity; and
an injector comprising a bracket to retain the hollow body of the device and at least one actuator of the piston, movably mounted with respect to the bracket, wherein said at least one actuator is configured to cooperate with the piston to move the piston within the receiving cavity.

2. The kit according to claim 1, wherein the piston comprises a distal body to obturate the cavity and a drive rod for the body, whereby the circulation channel passes through the distal body and through the rod.

3. The kit according to claim 1, wherein the assembly for selective distribution comprises a proximal distribution tap connected to the circulation channel opposite the receiving cavity and the proximal distribution tap can be moved jointly with the piston.

4. The kit according to claim 1, wherein the device comprises an auxiliary connection for evacuating the receiving cavity and the auxiliary connection opens into the receiving cavity away from the distal opening.

5. The kit according to claim 1, wherein the device comprises an evacuation line to be connected on the one hand to the receiving cavity and, on the other, to a negative pressure source.

6. The kit according to claim 1, wherein the device comprises at least one detachable sampling and/or reinjection line to be connected to the receiving cavity via the assembly for selective distribution.

7. The kit according to claim 1, wherein the proximal end of the hollow body is equipped with a lock suited to be received in a supplementary blocker of a centrifuge.

8. The kit according to claim 1, wherein the device comprises a reinjection line to be connected to the receiving cavity; the kit comprises an additional flow regulation device; and the additional device comprises an additional hollow body delimiting an intermediate volume, a piston that can be actuated manually, arranged on the additional hollow body, for downstream obturation of the intermediate volume, hereby the reinjection line is connected upstream to the receiving cavity) and downstream to the intermediate volume.

9. The kit according to claim 8, wherein the additional flow regulation device comprises a holder holding the manually actuated piston in place.

10. The kit according to claim 1, wherein the kit comprises a centrifuge, which comprises a housing for receiving the sampling device.

11. An injector configured to receive a device for sampling and reinjecting a fluid, comprising:
a hollow body delimiting a cavity for receiving the fluid, wherein the cavity opens in a proximal opening and at least one distal opening for sampling and/or reinjection; and
a piston, movably mounted in the receiving cavity to obturate the proximal opening;
wherein the device comprises an assembly for selective distribution of a proximal part of the fluid located in the receiving cavity, comprising a fluid circulation channel that passes through the piston, which channel opens into the receiving cavity;
wherein the injector comprises:
a retainer bracket for the hollow body of the device;
an actuator mounted movably with respect to the bracket, wherein the actuator is configured to cooperate with the piston to move the piston in the receiving cavity; and
wherein the actuator comprises a housing to receive the distribution assembly and/or a detachable line configured to be connected to the fluid circulation channel.

12. The injector according to claim 11, wherein the actuator comprises a rod that can be moved axially through the bracket, whereby the housing opens axially into a support surface on the piston and transversely through a lateral surface of the rod.

13. A non-therapeutic cosmetic treatment method for a living being, comprising:
providing a kit according to claim 1, whereby the receiving cavity of the body contains a non-therapeutic fluid; and
selectively evacuating a proximal part of the fluid present in the receiving cavity, whereby the piston obturates the proximal opening, independently of a distal part of the fluid located between the proximal part and the distal opening via the distribution assembly, the proximal part being evacuated through the evacuation channel arranged in the piston.

14. The method according to claim 13, whereby the method further comprises:
separating the fluid contained in the receiving cavity into a first proximal phase and at least one second distal phase; and
evacuating the first proximal phase from the receiving cavity through the distribution assembly without evacuating the second distal phase;

15. The method according to claim 13, wherein the device comprises a reinjection line to be connected to the receiving cavity; the kit comprises an additional flow regulation device; and the additional device comprises an additional hollow body delimiting an intermediate volume, a piston that can be actuated manually, arranged on the additional hollow body, for downstream obturation of the intermediate volume, hereby the reinjection line is connected upstream to the receiving cavity and downstream to the intermediate volume; and
wherein the method further comprises:
activating the injector to move fluid contained in the receiving cavity of the hollow body into an intermediate volume, and
manually actuating the piston in the additional hollow body to modify the flow rate of the fluid extracted from the additional flow regulation device.

16. The method according to claim 15, wherein said manually actuating comprises movement of the manually actuated piston in the additional hollow body to reduce the intermediate volume in order to increase the flow rate of the fluid extracted from the additional hollow body, or movement of the manually actuated piston in the additional hollow body to increase the intermediate volume in order to reduce the flow rate of the fluid extracted from the additional hollow body.

17. The kit according to claim 8, wherein the reinjection line is connected upstream to the receiving cavity) and downstream to the intermediate volume via the manually actuated piston.

18. The method according to claim 15, wherein the reinjection line is connected upstream to the receiving cavity) and downstream to the intermediate volume via the manually actuated piston.
